(19) United States
(12) Patent Application Publication
(10) Pub. No.: US 2003/0204317 A1
(43) Pub. Date: Oct. 30, 2003

(54) Methods, systems and software for displaying genomic sequence and annotations

(75) Inventors: Ann Loraine, El Cerrito, CA (US); Gregg Helt, Healdsburg, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/308,923

(22) Filed: Dec. 2, 2002

Related U.S. Application Data
(60) Provisional application No. 60/375,875, filed on Apr. 25, 2002.

(51) Int. Cl. G06F 19/00; G01N 33/48; G01N 33/50

(52) U.S. Cl. 702/20

(57) ABSTRACT

In some embodiments of the invention, methods, computer software products and computer systems are provided for displaying genetic information. In one embodiment, sermatic zooming is used to facilitate the viewing of genetic information.

### RELATED APPLICATIONS

This application claims the priority of U.S. Provisional Application No. 60/375,875, filed on Apr. 25, 2002. The '875 application is incorporated herein by reference for all purposes.

### BACKGROUND OF THE INVENTION

This invention is related to bioinformatics and biological data analysis and visualization. There is a great need in the art for genomic information analysis and visualization tools.

### SUMMARY OF THE INVENTION

This invention provides a computerized method of displaying genetic information and for displaying biological information. This invention additionally provides a computer software product for displaying genetic information and biological information and a system for displaying genetic information and biological information.

In one aspect of the invention, computer implemented methods of displaying genetic information are provided. In some embodiments, this method is accomplished by displaying genomic information on a computer display, receiving inputs from a user to change the magnification of the display, and displaying genomic information on the computer display using semantic zooming and where the magnification is determined according to the user's input. In one embodiment, the semantic zooming is one dimensional. The genomic information may include sequence information comprises sequence information displayed along a sequence axis. The result of zooming is typically the stretching of the sequence axis. The genomic information can be organized into a plurality of adjustable tiers. At least one of the adjustable tiers can be collapsed, moved, or hidden in response to user's input.

In another aspect of the invention, a computerized method for displaying biological information is provided. In some embodiments, the method is accomplished by displaying a representation of a genomic sequence on a computer display and displaying a representation of at least one protein motif corresponding to the genomic sequence on the computer display. In a preferred embodiment, a computerized method for displaying biological information includes receiving inputs from a user to change the magnification of the display and displaying the representation of the genome and the protein motif on the computer display using semantic zooming and where the magnification is determined according to the user's input.

In another aspect of the invention, a computer software product for displaying genetic information is provided. This computer software product is accomplished by a computer program code that performs the methods of the invention.

In yet another aspect of the invention, a system for displaying genetic information is provided. This system includes a processor, a memory being coupled to the processor, the memory storing a plurality of machine instructions that cause the processor to perform a plurality of logical steps that performs the methods of the invention.

### BRIEF DESCRIPTION OF THE DRAWINGS

FIG. 1 is an annotated GeneViewer screen capture showing FLJ22324, a six-exon gene inferred from a cDNA-to-genomic sequence alignment. (a) The high-level structure at low zoom. (b) A close-up view of a questionable small intron separating exons 5 and 6. Dinucleotide bases at this intron's 5′ and 3′ boundaries are underlined.

FIG. 2 illustrates how semantic zooming could be used to represent gene structure annotations based on cDNA-to-genomic sequence alignments. (a) Low zoom. (b) High zoom.

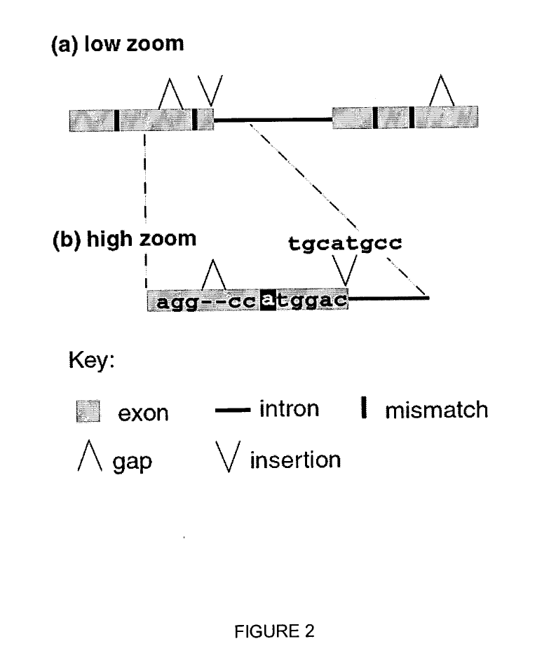

FIG. 3 shows SNURF locus. (a) The full scene is shown with multiple annotation types sorted into labeled tiers. (b) A simplified scene is shown in which several tiers shown in (a) have been hidden, collapsed, or moved to new positions. The horizontal slider has been used to expand the display in the vertical direction.

FIG. 4 shows using color to represent frame of translation at the ARG1 locus. Coding regions in each exon are colored according to which frame of the genomic sequence is translated. A different color for overlapping exons from different transcripts indicates these exons are translated in different frames.

FIG. 5 shows protein motifs detected by Pfam displayed beneath alternative transcript structures (a,b) at the PLAT locus. Alignments between genomic sequence and cDNA sequences (a) BC002795.1 and (b) NM_{—}0009301 are shown. Regions encoding matches to Pfam motifs PF00008 (EGF-like domain), PF00051 (Kringle), PF00089 (Serine proteases, trypsin family), and PF00039 (Type I fibronectin) are shown as linked green rectangles below each alignment.

### DETAILED DESCRIPTION

Reference will now be made in detail to the exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

Throughout this disclosure, various publications, patents and published patent specifications are referenced by an identifying citation.

Throughout this disclosure, various aspects of this invention may be presented in a range format. It should be understood that the description in range format is merely for convenience and brevity and should not be construed as an inflexible limitation on the scope of the invention.

The practice of the present invention will employ, unless otherwise indicated, conventional techniques of bioinformatics, computer sciences, immunology, biochemistry, chemistry, molecular biology, microbiology, cell biology, genomics and recombinant DNA, which are within the skill of the art. See, e.g., Setubal and Meidanis, et al., 1997, Introduction to Computational Molecular Biology, PWS Publishing Company, Boston; Human Genome Mapping Project Resource Centre (Cambridge), 1998, Guide to Human Genome Computing, 2nd Edition, Martin J. Biship (Editor), Academic Press, San Diego; Salzberg, Searles, Kasif, (Editors), 1998, Computational Methods in Molecular Biology, Elsevier, Amsterdam; Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987))

One of skill in the art would appreciate that many computer systems are suitable for carrying out the methods of the invention. Computer software according to the embodiments of the invention can be executed in a wide variety of computer systems.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

Computer software products of the invention typically include computer readable medium having computer-executable instructions for performing the logic steps of the methods of the invention. Suitable computer readable medium include floppy disk, CD-ROM/DVD/DVD-ROM, hard-disk drive, flash memory, ROM/RAM, magnetic tapes and etc. The computer executable instructions may be written in any suitable computer language or combination of several languages. Suitable computer languages include C/C++ (such as Visual C/C++), C#, Java, Basic (such as Visual Basic), SQL, Fortran, SAS and Perl.

In one aspect of the invention, several techniques for presenting human genomic sequence data and annotations in an interactive, graphical format. The methods, systems and computer software products provided by the inventions described herein have extensive practical applications such as drug discovery and research, microarray design, etc

The goal of the publicly funded human genome project is to provide biologists with a reference human genome sequence and in so doing, accelerate the pace of biomedical research. In addition to providing this reference sequence, the Human Genome Consortium has also provided initial interpretation of the raw sequence data in the form of annotations, notations on the sequence data which describe the location of biologically meaningful features embedded in the data. Thus far, these feature annotations have included three basic types: single-base annotations such as such as the location of single-nucleotide polymorphisms (SNPs), single-span annotations such as the location and extent of individual transposable elements, and multi-span annotations such as the locations of a gene's complement of exons and introns as inferred from cDNA-to-genomic sequence alignments or predicted by gene-finding programs such as GenScan (C. Burge and S. Karlin, “Prediction of complete gene structures in human genomic DNA,” J Mol Biol, vol. 268, pp. 78-94, 1997) and Genie (D. Kulp, D. Haussler, M. G. Reese, and F. H. Eeckman, “A generalized hidden Markov model for the recognition of human genes in DNA,” Proc Int Conf Intell Syst Mol Biol, vol. 4, pp. 134-42, 1996).

These location-based feature annotations often possess annotations of their own, such as scores describing their believability, information about the analysis programs used to generate them, their type, and other descriptive data. Although the basic sequence data are valuable and useful, biologists are typically more interested in the higher-level, location-based annotations on the sequence, since these annotations relate the sequence data to biological pathways and systems. All three levels of genomic data can be described using a simple text-based format, but biologists can make sense of the information more effectively when it is presented in an interactive, graphical format.

In recognition of the value of graphical representation, genomic data providers have developed graphical tools which generally follow a Web-based, client-server model, in which server-side programs create image-mapped genomic “scenes” that are then displayed in the user's browser. Two prominent examples of this Web-based approach include NCBI's evidence viewer (www.ncbi.nlm.nih.gov) and the UCSC genome gateway's genome browser (genome.ucsc.edu/cgi-bin/hgGateway). These and similar Web sites provide valuable services, but are limited by the inability of a client-server model to provide a truly interactive user experience, since each interaction with the genome scene on display requires a round-trip from the user's desktop to the server and back again. Furthermore, the image-map format is currently unable to support gestural interactions, such as dragging scrollbars to pan or change the magnification.

When exploring a genomic region, biologists need to interact with the scene in a much richer fashion than is currently possible using simple, hyperlinked images. As biologists begin to explore a genomic scene, they formulate new questions about what they see. In order to answer these questions, they often need to modify what they see, such as by adding or deleting data, panning to the left or right, or changing the scale of the view.

The client-server model is limited because it constrains navigation, often requiring multiple clicks to make the simplest adjustments. Hybrid approaches such as interactive, Java applets downloaded from a server and which run in a Java virtual machine on the user's desktop, have also been attempted (Helt G A, Lewis S, Loraine A E, Rubin G M: BioViews: Java-based tools for genomic data visualization. Genome Res 1998, 8:291-305), but Java applets are restricted in their ability to load data files located on the user's personal computer, because of security concerns. Thus to gain the full benefit of genome project data, users require desktop software that can present the data in a fully interactive environment conducive to exploration and which also allows users to view their own custom data.

In one aspect of the invention, methods, systems and software are provided for presenting human genomic sequence data and annotations in an interactive, graphical format.

Representing Gene Structures

Representing gene structures is one crucial aspect of any genome browser application. In one aspect of the invention, methods, software and systems for representing the key elements of human gene structures and their encoded proteins are provided. A gene structure, as used here, is defined as the relative placement of feature elements that make up a gene onto a single linear axis defined by the DNA sequence. Feature elements that make up a gene include: exons, 5′ and 3′ untranslated regions, coding regions, start and stop codons, introns, 5′ transcriptional control elements, 3′ polyadenylation signals, splice site boundaries, and protein-based annotations of the coding regions. Each of these feature types may require specialized methods of presentation that depend upon the type of data being shown.

Visualizing Sequence Using Semantic Zooming

Although gene structures and their annotations are mainly what interest biologists, the ability to view the sequence data in the context of a gene structure is a critical feature of any genome browser application (Helt G A, Lewis S, Loraine A E, Rubin G M: BioViews: Java-based tools for genomic data visualization. Genome Res 1998, 8:291-305). In order to interpret and assess a proposed gene structure, biologists need to be able to inspect individual bases that influence the gene structure's believability. For example, to assess whether the reported 3′ end of a gene is correct, a biologist may wish to search the sequence near the end of the final exon for putative polyadenylation sites. Similarly, a biologist may wish to examine the dinucleotide sequence at the 5′ and 3′ boundaries of putative introns, since these bases are highly conserved in human genes (Lander E S, Linton L M, Birren B, Nusbaum C, Zody M C, Baldwin J, Devon K, Dewar K, Doyle M, FitzHugh W, et al.: Initial sequencing and analysis of the human genome. Nature 2001, 409:860-921).

In one aspect of the invention, methods, systems and computer software are provided for visualizing genomic information. In some embodiments, semantic zooming is used to display genomic information.

In semantic zooming, objects may change appearance or shape as they change size. For example, a growing dot may become a simple box, then a box with a one-word label, then a box with a longer label, then a rectangle filled with text and pictures. The goal is to give the most meaningful presentation at each size. For a review of semantic and other zooming technology, see, e.g., CounterPoint: Creating Jazzy Interactive Presentations, Good, L., Bederson, B. B., HCIL-2001-3, CS-TR-4225, UMIACS-TR-2001-14, March 2001; Jazz: An Extensible Zoomable User Interface Graphics Toolkit in Java, Bederson, B., Meyer, J., Good, L. HCIL-2000-13, CS-TR-4137, UMIACS-TR2000-30, May 2000, In ACM UIST 2000, pp. 171-180; Jazz: An Extensible 2D+ Zooming Graphics Toolkit in Java Bederson, B., McAlister, B. HCIL-99-07, CS-TR-4015, UMIACS-TR-99-24, May 1999; Does Zooming Improve Image Browsing? Combs, T., T. A., and Bederson, B., HCIL-99-05, CS-TR-3995, UMIACS-TR-99-14, February 1999 In ACM Digital Library Conference, pp. 130-137; Graphical Multiscale Web Histories: A Study of PadPrints Hightower, R. R., Ring, L. T., Helfman, J. I., Bederson, B. B., and Hollan, J. D. ACM Conference on Hypertext 1999; Does Animation Help Users Build Mental Maps of Spatial Information, Bederson, B. and Boltman, A., CS-TR-3964, UMIACS-TR-98-73, September 1998, In IEEE Info Vis 99, pp. 28-35; A Zooming Web Browser, Bederson, B. B., Hollan, J. D., Stewart, J., Rogers, D., Vick, D., Ring, L. T., Grose, E., Forsythe, C.. Human Factors in Web Development, Eds. Ratner, Grose, and Forsythe, Lawrence Erlbaum Assoc., pp 255-266, 1998; Implementing a Zooming User Interface: Experience Building Pad++, Bederson, B., Meyer, J., Software: Practice and Experience, 28 (10), pp. 1101-1135, August 1998; When Two Hands Are Better Than One:Enhancing Collaboration Using Single Display Groupware, Stewart, J., Raybourn, E. M., Bederson, B. B., Druin, A., ACM CHI 98 Summary, 1998; KidPad: A Design Collaboration Between Children, Technologists, and Educators, Druin, A., Stewart, J., Proft, D., Bederson, B. B., Hollan, J. D., ACM CHI 97, pp 463-470, 1997; A Multiscale Narrative: Gray Matters, Wardrip-Fruin, N., Meyer, J., Perlin, J., Bederson, B. B., Hollan, J. D.,ACM SIGGRAPH 97 Visual Proceedings, p 141, 1997; A Zooming Web Browser, Bederson, B. B., Hollan, J. D., Stewart, J., Rogers, D., Druin, A., and Vick, D. SPIE Multimedia Computing and Networking, Volume 2667, pp 260-271, 1996; Local Tools: An Alternative to Tool Palettes, Bederson, B. B., Hollan, J. D., Drum, A., Stewart, J., Rogers, D., Proft, D., ACM UIST '96, pp 169-170, 1996; Pad++: A Zoomable Graphical Sketchpad for Exploring Alternate Interface Physics, Bederson, B., Hollan, J., Perlin, K., Meyer, J., Bacon, D., and Furnas, G., Journal of Visual Languages and Computing, 7, 3-31, 1996, HTML, Postscript without pictures (74K), PDF without pictures (77K) 1995; Space-Scale Diagrams: Understanding Multiscale Interfaces, Furnas, G., Bederson, B., ACM SIGCHI '95; Advances in the Pad++ Zoomable Graphics Widget, Bederson, B., Hollan, J. USENIX Tcl/Tk'95 Workshop; Pad++: Advances in Multiscale Interfaces, Bederson, B. B., Stead, L., Hollan, J. D. ACM SIGCHI '94 (short paper), 1994; Pad++: A Zooming Graphical Interface for Exploring Alternate Interface Physics, Bederson, B. B., Hollan, J. D., , ACM UIST '94, 1994; Pad—An Alternative Approach to the Computer Interface, Perlin, K., Fox, D., ACM SIGGRAPH '93; A Multiscale Approach to Interactive Display Organization, Perlin, K., Coordination Theory and Collaboration Technology Workshop, National Science Foundation, June 1991.

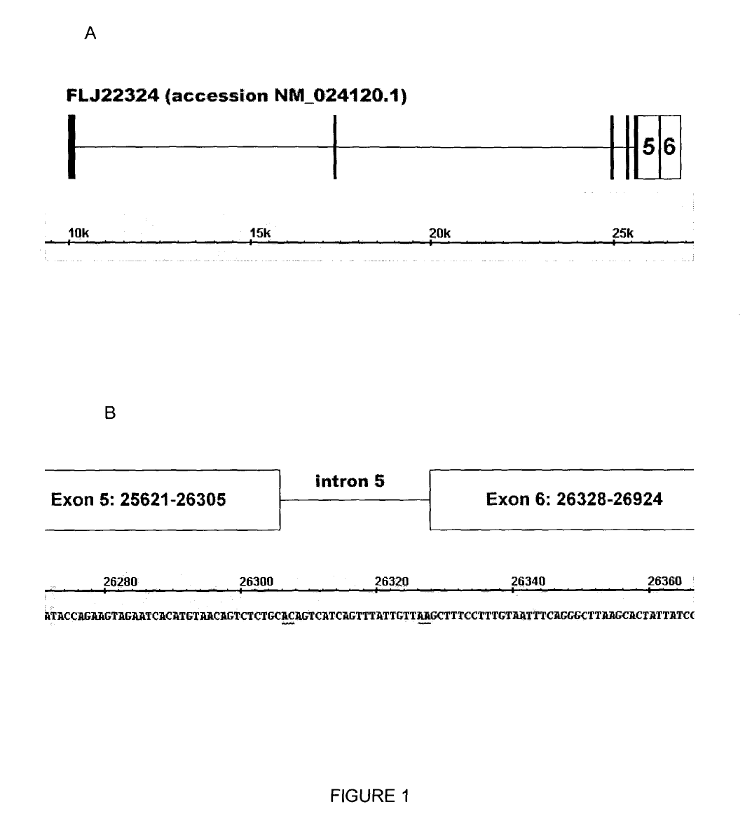

FIG. 1 presents an example of how semantic zooming, a visualization technique in which objects change their representation according to their level of magnification (zoom level), can be used to convey sequence information alongside a representation of a gene structure. In this example, a human gene structure has been inferred from an alignment between a single cDNA sequence (Genbank accession NM_{—}024120) and its putative genomic region of origin. The pattern of aligned spans, represented as tall rectangles, defines a simple gene structure containing a very small intron interrupting its 3′ untranslated region Since the average size for a 3′ UTR intron is about 100 times larger than this intron, a biologist attempting to evaluate this gene might doubt whether this apparent intron is real. In one embodiment, the intron boundaries are examined at the level of individual bases, since the sequence of the intron boundaries can give an indication of whether this intron is believable.

FIG. 1(a) shows the alignment at low magnification, while FIG. 1(b) shows how the same scene would look at higher level of magnification. The low magnification view shows the gross structure of the gene, while the higher magnification view shows both the boundaries of the questionable intron together with the genomic sequence. In this case, the close-up view in FIG. 1(b) reveals that the bases flanking the intron deviate from the expected “gt--intron--ag” consensus sequence for intron boundaries, thus lending credence to the idea that this insertion in the genomic sequence relative to the aligned cDNA may represent an experimental artifact.

In this example, changing the scale of the display (zooming “in”) allows the user to inspect the genomic sequence while at the same time maintaining a sense of context. In other words, when looking at individual bases at high magnification, the user is able to maintain a sense of place since the sequence is shown alongside the same landmarks that were visible at low zoom.

Another use of semantic zooming involves labeling or otherwise annotating display elements with text. For example, at low zoom, an object may be too small to fit a label, such as the 5′ exons in FIG. 1a. At high zoom, objects take over more screen real estate and therefore become large enough to fit progressively longer labels. Thus semantic zooming allows the primary sequence data, location-based features on the sequence, and text-based annotations on these features to be presented together in a single highly adjustable scene.

One Versus Two Dimensional Zooming

Zooming as a visualization technique for display and navigation of complex data sets has typically been implemented in two dimensions. For example, applications built using the Java-based Jazz toolkit for graphical user interfaces (Bederson B, Myer J, Good L: Jazz: an extensible zoomable user interface graphics toolkit in Java. In: ACM UIST; 2000; San Diego, Calif. 171-181) provide point-based or “camera” zooming, in which the operation of zooming is best understood as a change in height of a virtual camera poised above a single point in the display. That is, in camera-based zooming, the entire scene appears to expand or contract in every direction around a central focal point as the user zooms in or out.

Genome browser applications typically represent a one-dimensional world in that they display location-based features across a single defined by the genomic sequence data itself. Thus, in some instances, it may be appropriate for genome browsers to restrict zooming to the same dimension as the sequence axis. In contrast, applications built using the Java-based Jazz toolkit for graphical user interfaces (B. Bederson, J. Myer, and L. Good, “Jazz: an extensible zoomable user interface graphics toolkit in Java,” presented at ACM UIST, San Diego, Calif., 2000) provide point-based or “camera” zooming, in which the operation of zooming is best understood as a change in height of a camera poised above a single point in the display. That is, in camera-based zooming, the entire scene appears to expand or contract in every direction around a central focal point as the user zooms in or out.

In genome browser applications, camera-based, point-centered zooming as provided in Jazz may not be appropriate for some embodiments, since DNA sequence and its annotations are one-dimensional. In genome browser type applications, the axis perpendicular to the sequence axis may have no meaning in some case except perhaps as a convenient way to sort information, as will be discussed later. As with Jazz-based applications, the focus for zooming should still be the point where the user last clicked, but the result of zooming should be a stretching of the sequence axis rather than a change in the user's relative height above the genomic scene.

Single and Dual-Sequence Annotations

Gene structures are typically deduced using one of two methods. The simplest type of gene structure is based on output from gene-finding programs that analyze the primary genomic sequence without reference to any other sequence. For example, gene prediction programs produce simple gene structures based solely on analysis of primary genomic sequence data. These simple annotations may easily be shown as linked, multi-span annotations representing the complement of exons that make up these hypothetical gene structures.

In contrast, a dual-sequence annotation describes a relationship between the genomic sequence and the independently produced sequence of some other biological molecule. For example, programs such as sim4 (L. Florea, G. Hartzell, Z. Zhang, G. M. Rubin, and W. Miller, “A computer program for aligning a cDNA sequence with a genomic DNA sequence,” Genome Res, vol. 8, pp. 967-74., 1998), and blat (W. J. Kent, “BLAT-the BLAST-like alignment tool,” Genome Res, vol. 12, pp. 656-64., 2002.) designed to align cDNA with genomic sequences are often used to infer gene structures in genomic DNA. Regions in the genomic sequence that match a region in the cDNA sequence typically are used to delimit exons, while gaps that exceed some pre-defined length threshold are typically used to delimit introns.

Although sequence analysis programs are useful tools, their ability to accurately describe human gene structures is severely hampered by the complex nature of human genes (E. S. Lander, et al., “Initial sequencing and analysis of the human genome,”Nature, vol. 409, pp. 860-921., 2001). Approximately a third to over half of all human genes produce multiple transcript variants (E. S. Lander, et al., “Initial sequencing and analysis of the human genome,” Nature, vol. 409, pp. 860-921., 2001; A. A. Mironov, J. W. Fickett, and M. S. Gelfand, “Frequent alternative splicing of human genes,” Genome Res, vol. 9, pp. 1288-93., 1999), and few gene prediction programs are able to identify more than one variant per gene. Because gene-finding programs are still limited in their ability to accurately describe human gene structures, applications that display them should make their hypothetical nature clear to the user.

In contrast, a dual-sequence or pairwise annotations describe the relationship between the genomic sequence and the independently produced sequence of some other biological molecule. For example, programs such as sim4 (Florea L, Hartzell G, Zhang Z, Rubin G M, Miller W: A computer program for aligning a cDNA sequence with a genomic DNA sequence. Genome Res 1998, 8:967-74) and blat (Kent W J: BLAT-the BLAST-like alignment tool. Genome Res 2002, 12:656-64.) are designed to align cDNA and genomic sequence and are often used to infer gene structures in genomic DNA. Regions in the genomic sequence that match regions in the cDNA sequence typically are used to delimit exons, while gaps in the cDNA partner of the alignment that exceed some pre-defined length threshold are typically used to delimit introns.

Annotations like these are often more valuable than simple gene predictions because they incorporate more information, such as experimental evidence for expression provided by cDNA sequence. And because they incorporate more information, their representation in a genome browser requires a more sophisticated approach that shows the exon-intron organization of the inferred gene structure as well as the alignment that is used to produce it.

Dealing with Complexity

The number and type of annotations can vary enormously from region to region depending both on the sequence itself as well as the number and types of analyses that have been done. Thus, it is difficult to design a program that can arrange sequence-based annotations in a fashion that is not overly confusing or complex. However, since genomic sequence has only one important axis, which can be shown either in vertical or horizontal orientation, the application developer can use the other axis to organize information in ways that expose biologically meaningful patterns in the data, such as regions that are densely annotated with ESTs and likely to be highly expressed.

One commonly-used technique for dealing with complexity of genomic annotations is to sort items into horizontal tiers based on some common attribute, such as the kind of analysis that was done to produce them. This approach is being used by the U.C.S.C. genome browser, which provides multiple rows or “tracks” featuring analyses contributed by many different groups (Kent W J, Sugnet C W, Furey T S, Roskin K M, Pringle T H, Zahler A M, Haussler D: The human genome browser at UCSC. Genome Res 2002, 12:996-1006.).

Even so, as the available analyses accumulate, the potential for creating very complex scenes increases greatly. Although it is good to have more access to more data, for the purposes of understanding a gene it is important to give the user the power to simplify or re-organize the scene as needed. Sorting items into distinct tiers that can be moved, hidden, collapsed, or stretched is one way that application developers can give users greater freedom to modify a display when the amount of data being shown exceeds the user's ability to comprehend the full scene.

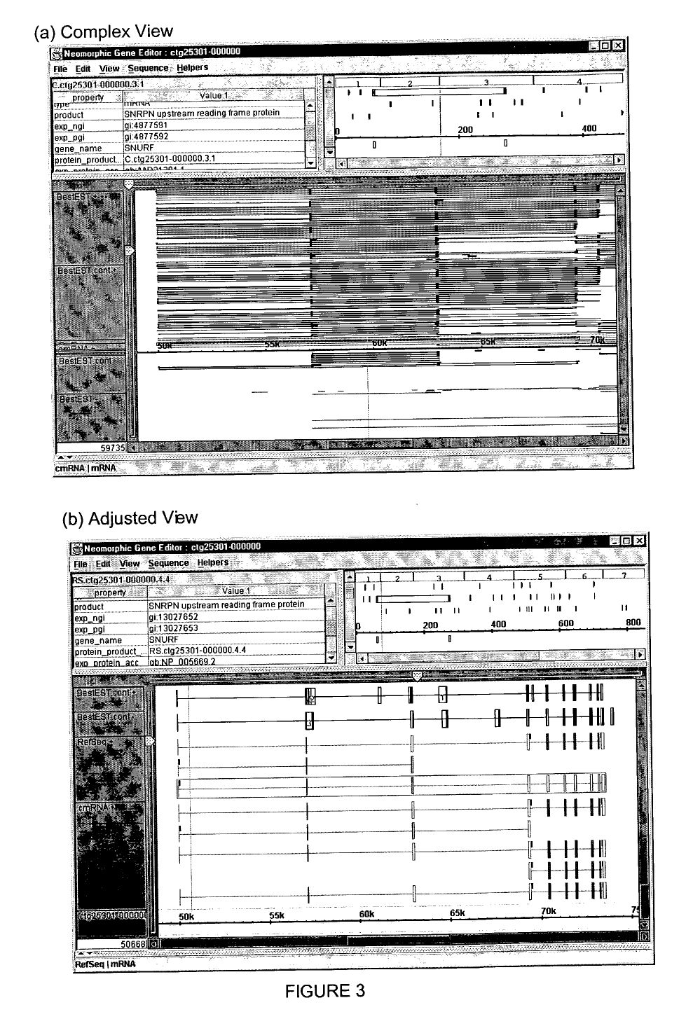

An example of a complex scene organized into adjustable tiers is shown in FIG. 3, which shows two screen captures from the Gene Viewer display tool. Both screen captures present a view of the human SNURF gene, which gives rise to an unusual bicistronic transcript encoding two different proteins (Gray T A, Saitoh S, Nicholls R D: An imprinted, mammalian bicistronic transcript encodes two independent proteins. Proc Natl Acad Sci USA 1999, 96:5616-21). FIG. 3a presents a complex view of the locus, shown here as annotated with hundreds of features derived from EST-to-genomic and cDNA-to-genomic sequence alignments.

The screen capture in FIG. 3b shows a simpler version in which the display has been simplified by collapsing, moving, and hiding several tiers. The tiers containing features based on EST-to-genomic sequence alignments have been collapsed, thus forming primitive clusters summarizing all the expressed regions associated with this locus. The plus- and minus-strand EST tiers have also been moved to the position immediately above the tiers showing cDNA-to-genome alignments (labeled cmRNA+ and RefSeq+), thus making it easier to compare the boundaries of items shown in each tier. This feature is especially important to users interested in alternative splicing, since the presence of ESTs that align to regions not covered by cDNA-to-genomic alignments can indicate the existence of novel variants.

Another way to help users visualize a genomic scene using tiers is to allow the user to stretch tiers in the direction perpendicular to the sequence axis. Allowing zooming in the vertical direction, as is shown in FIG. 3b, accommodates situations where the entire scene cannot fit into the viewable area. In addition, this kind of vertical stretching allows users with visual disabilities to make selected regions of the scene larger and easier to see.

Protein in the Context of Genomic Sequence

Although the intron-exon organization of genes is interesting, biologists typically are more interested in the proteins that genes encode. Therefore a genomic viewer should incorporate information describing how the genomic sequence is translated into protein.

One common convention for representing coding regions is to show translated and untranslated regions as shaded and unshaded boxes, respectively. This convention has been used for many years to represent coding regions in the print medium and therefore has the advantage of leveraging users' expectations of how gene structures should look when presented in software.

Merely indicating the translated regions is not sufficient, however, because many genes give rise to multiple transcript forms due to alternative splicing, alternative promoter choice and other mechanisms that generate transcript and protein diversity. In many cases, alternative transcript structure causes shifts in frame. To accommodate this, it is provided in accordance with some implementations of the present invention that genome browsers use shading or color to indicate the frame of translation for each coding exon.

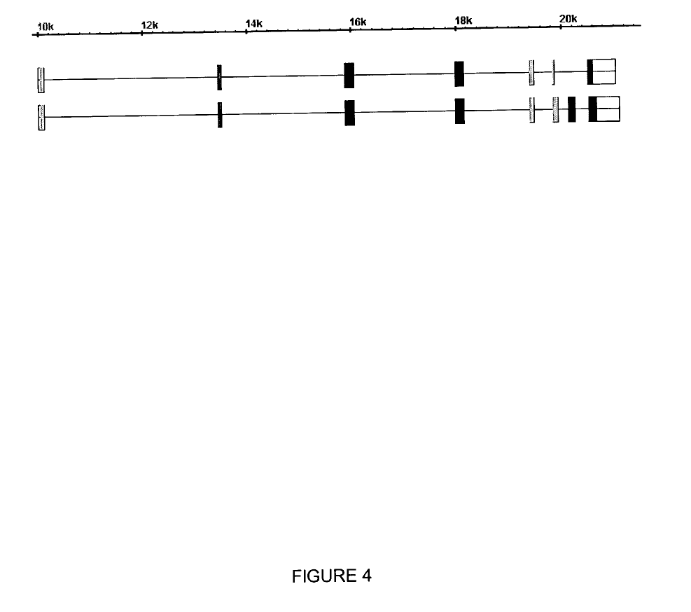

FIG. 4 shows an example of how shading reveals the differences between two distinct transcript variants encoded at the AIRE (auto-immune regulator) locus. The bottom variant contains an additional 3′ exon which causes a frameshift in the final exon relative to the top variant. Thus although the final exon in both variants begins at the same position, differences in upstream splicing result in this exon encoding two different peptides in the two different variants. Such examples are common among human genes, and therefore understanding alternative transcript structure requires a depiction of translation frame in addition to the relative placement of introns and exons.

In addition to providing a visual representation of how genomic sequence is translated into protein, a genome browser application should also provide a visual representation of motifs that are embedded in the protein sequence. In recent years, numerous protein sequence analysis methods have been developed that allow researchers to identify conserved functional domains within protein sequence. Since alternative transcripts arising from the same gene can often produce protein isoforms that differ with respect to their domain composition, tools that display these protein domains in the context of the genomic structure of genes can help biologists understand how alternative transcript structure affects protein function.

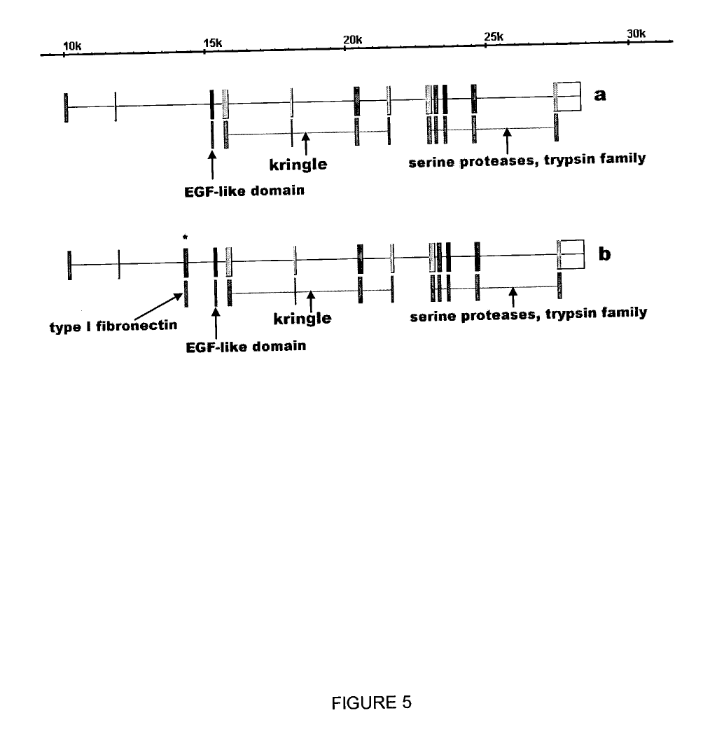

FIG. 5 presents a visualization of the PEG3 locus (paternally expressed 3) which encodes two distinct protein isoforms that differ with respect to their domain composition. The shorter variant (Genbank accession AF208967.1) contains multiple C-terminal Zn finger motifs in addition to an upstream SCAN domain. The longer isoform (Genbank accession AAF6178.1) lacks the SCAN domain, a result of alternative splicing. Like the shorter form, it contains C-terminal Zn finger motifs, but fewer than the shorter form. It also contains a KRAB motif upstream of the C-terminus which is not present in the shorter form. Since all three domains are involved in transcriptional activation, it is likely that these two proteins serve distinct functions in transcriptional regulation. Thus a full understanding of the PEG3 locus requires visualization of the alternative transcripts this gene encodes as well as the disparate domains present in each protein isoform.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the invention.

What is claimed is:

1. A computerized method of displaying genetic information comprising:
Displaying genomic information on a computer display;
Receiving inputs from a user to change magnification of the display; and
Displaying genomic information on the computer display using semantic zooming wherein the magnification is determined according to the user's input.

2. The method of claim 1 wherein the semantic zooming is one dimensional.

3. The method of claim 2 wherein the genomic information comprises sequence information displayed along a sequence axis.

4. The method of claim 3 wherein the semantic zooming is along the sequence axis.

5. The method of claim 4 wherein the result of zooming is the stretching of the sequence axis.

6. The method of claim 1 wherein the genomic information is organized into a plurality of adjustable tiers.

7. The method of claim 6 wherein at least one of the adjustable tiers can be collapsed, moved, or hidden in response to user's input.

8. A computerized method for displaying biological information comprising:
Displaying a representation of a genomic sequence on a computer display; and
Displaying a representation of at least one protein motif corresponding to said genomic sequence on the computer display.

9. The method of claim 8 further comprising:
Receiving inputs from a user to change the magnification of the display; and
Displaying the representation of the genome and the protein motif on the computer display using semantic zooming and where the magnification is determined according to the user's input.

10. The method of claim 9 wherein the semantic zooming is one dimensional.

11. The method of claim 10 wherein the genomic information comprises sequence information displayed along a sequence axis.

12. The method of claim 11 wherein the semantic zooming is along the sequence axis.

13. The method of claim 12 wherein the result of zooming is the stretching of the sequence axis.

14. A computer software product for displaying genetic information comprising:
Computer program code that displays genomic information on the computer display;
Computer program code that inputs from a user to change the magnification of the display;
Computer program code that displays genomic information on the computer display using semantic zooming and where the magnification is determined according to the user's input, and
A computer readable medium that stores the codes.

15. The computer software product of claim 14, wherein the semantic zooming is one dimensional.

16. The computer software product of claim 15, wherein the genomic information comprises sequence information displayed along the sequence axis.

17. The computer software product of claim 16, wherein the semantic zooming is along the sequence axis.

18. The computer software product of claim 17, wherein the result of zooming is the stretching of the sequence axis.

19. The computer software product of claim 18, wherein the genomic information is organized into the plurality of adjustable tiers.

20. The computer software product of claim 19, wherein at least one of the adjustable tiers can be collapsed, moved, or hidden in response to user's input.

21. A computer software product displaying biological information comprising:
Computer program code that displays the representation of a genomic sequence on the computer display;
Computer program code that displays a representation of at least one protein motif corresponding to the genomic sequence; and
A computer readable medium that stores the codes.

22. The computer software product of claim 21 further comprising
Computer program code that receives inputs from a user to change the magnification of the display; and
Computer program code that displays the representation of the genome and the protein motif on the computer display using semantic zooming and where the magnification is determined according to the user's input.

23. The computer software product of claim 22, wherein the semantic zooming is one dimensional.

24. The computer software product of claim 23, wherein the genomic information comprises sequence information displayed along the sequence axis.

25. The computer software product of claim 24, wherein the semantic zooming is along the sequence axis.

26. The computer software product of claim 25, wherein the result of zooming is the stretching of the sequence axis.

27. A system for displaying genetic information comprising:
A processor;
A memory being coupled to the processor, the memory storing a plurality of machine instructions that cause the processor to perform a plurality of logical steps when implemented by the processor, said logical steps including:
Displaying genomic information on a computer display;
Receiving inputs from a user to change the magnification of the display; and
Displaying genomic information on the computer display using semantic zooming and where the magnification is determined according to the user's input.

28. The system in claim 27, wherein the semantic zooming is one dimensional.

29. The system in claim 28, wherein the genomic information comprises sequence information displayed along the sequence axis.

30. The system in claim 29, wherein the semantic zooming is along the sequence axis.

31. The system in claim 30, wherein the result of zooming is the stretching of the sequence axis.

32. The system of claim 31, wherein the genomic information is organized into the plurality of adjustable tiers.

33. The system of claim 32, wherein at least one of the adjustable tiers can be collapsed, moved, or hidden in response to user's input.

34. A system for displaying genetic information comprising:
A processor;
A memory being coupled to the processor, the memory storing a plurality machine instructions that cause the processor to perform a plurality of logical steps when implemented by the processor, said logical steps including:
Displaying a representation of a genomic sequence on the computer display; and
Displaying a representation of at least one protein motif corresponding to said genomic sequence on the computer display.

35. The system of claim 34, further comprising:
Receiving inputs from a user to change the magnification of the display; and
Displaying the representation of the genome and the protein motif on the computer display using semantic zooming and where the magnification is determined according to the user's input.

36. The system of claim 35, wherein the semantic zooming is one dimensional.

37. The system of claim 36, wherein the genomic information comprises sequence information displayed along a sequence axis.

38. The system of claim 37, wherein the semantic zooming is along the sequence axis.

39. The system of claim 38, wherein the result of zooming is the stretching of the sequence axis.
